(19) United States
(12) Patent Application Publication
(10) Pub. No.: US 2004/0049139 A1
(43) Pub. Date: Mar. 11, 2004

(54) Therapeutic lower extremity device

(76) Inventor: Marin Craciunescu, Seven Hills, OH (US)

Correspondence Address: MARIN CRACIUNESCU, 4234 VEZBER DRIVE, SEVEN HILLS, OH, 44131, US

(21) Appl. No.: 10/375,703

(22) Filed: Feb. 26, 2003

Related U.S. Application Data
(60) Provisional application No. 60/408,912, filed on Sep. 5, 2002.

(51) Int. Cl. A61F 5/00

(52) U.S. Cl. 602/13

(57) ABSTRACT

A therapeutic lower extremity device is provided. The device generally comprises a pair of coupling members 10 and 12, interconnected by a tensioner structure 14. The tensioner structure 14 enables an adjustable linear displacement between the coupling members 10 and 12, and may include a goniometer arrangement 16 for angle readings between the coupling members when applied on a patient. A pair of air inflatable elements 30 and 30A are provided in apposition to each other in each of the coupling members. The air inflatable elements are preferably connected to corresponding air pressure gauges 36 to facilitate a reading of the muscle contraction strength.

The present device may be employed in the rehabilitation process of the lower or upper extremities in an essentially isometric manner.

### CROSS-REFERENCE TO RELATED APPLICATIONS

PPA Ser. No. 60/408,912 Filed Sep. 5, 2002

### FIELD OF THE INVENTION

This invention relates to therapeutic portable exercise devices and braces for conditioning and strengthening the muscles and tendons in the lower extremity of a person.

### BACKGROUND OF THE INVENTION

Lower extremity injuries and other types of debilitating illnesses frequently require prolonged immobilization which tends to cause general weakening of the muscles in the human body. The use of lower extremity therapeutic devices of known design and configuration is known in prior art.

Known prior art includes U.S. Pat. Nos. 6,409,393 B1; 6,296,595B1 and 6,336,909B2. While these devices fulfill their respective particular objectives and requirements, the aforementioned patents are limited in the therapeutic applications of the lower extremity.

### SUMMARY OF THE INVENTION

The general purpose of the present invention is to provide the rehabilitation professionals with a versatile simple and inexpensive device for rehabilitation of an affected lower extremity. A person may exercise with this device in a supine or sitting position.

To attain this preferably the present invention comprises a pair of coupling members or braces for positioning around the lower extremities of a person above the knees. A tensioner structure which includes a goniometer arrangement is extended between and connected to each of the braces or coupling members. Each coupling member typically includes a plurality of inflatable elements positioned in such a manner as to restrict an unwanted displacement of the lower extremity when the affected muscle groups are contracted. In addition, a plurality of hook and loop fasteners are positioned to secure a comfortable fit of the coupling members around the lower extremities.

Also this device may be employed in the therapeutic rehabilitation of the upper extremities.

Alternatively, the present invention may be employed as a brace arrangement to assist in decreasing of lower or upper extremities contractures in the rehabilitation process.

### OBJECTS AND ADVANTAGES

Accordingly, it can be appreciated that there exists a continuing need for a new more versatile lower extremity therapeutic device which is versatile and easy to handle in the process of human rehabilitation.

Therefore, several objects and advantages of the present invention are:

a. To provide a therapeutic device for strengthening of the affected lower extremity in an isometric manner. It is known that muscles should be exercised to maintain strength. It is also known that there are circumstances where specific joints are restricted to move due to surgical procedures or healing fractures. In most of these cases the physician restricts the joint movement but allows active muscle contractures. Therefore, when a muscle contracture takes place without a corresponding joint movement an isometric muscle contraction takes place.

b. To provide an easy to handle lower extremity therapeutic device, that can be comfortable used in a supine or in a sitting position.

c. To provide a brace arrangement to assist in decreasing of lower extremities contractures. With the present invention, decreased hands on therapy is needed since the patient will be able to perform the prescribed exercises with less supervision. Furthermore, both the patient and therapist will benefit from an accurate feed back of the muscles contraction strength by reading the included air pressure gauges. Especially in the therapeutic rehabilitation of a stroke patient when a lower extremity is weaker than the other, it will be appreciated that the muscular activity may be measured in a very accurate manner and the therapeutic progress may be objectively recorded.

### BRIEF DESCRIPTION OF THE DRAWING

FIG. 1 is a front view of a therapeutic lower extremity device in accordance with the present invention.

FIG. 2 illustrates a longitudinal top view of the tensioner structure in combination with a goniometer arrangement.

FIG. 2A shows a longitudinal side view of the tensioner structure in combination with a goniometer arrangement.

FIG. 3 is a diagrammatic representation of the air pressure arrangement included in the present invention

FIG. 4 is a lateral side view of the present invention

### DETAILED DESCRIPTION OF THE DRAWINGS

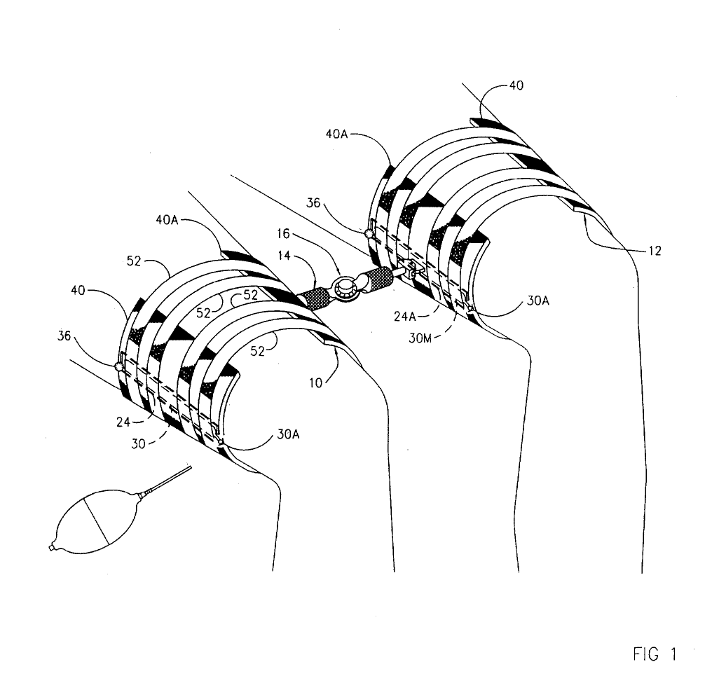

FIG. 1 illustrates the front view of the present invention. It mainly consists of a pair of spaced apart coupling members or braces 10 and 12 constructed to be removably positioned around the lower extremities of a person.

Each of the coupling members 10 and 12 may include any combination of components such as fully-, non-, or partially stretchable fabric, soft foam, hook and loop fasteners, and any other suitable elements. The coupling members 10 and 12 are interconnected by a tensioner arrangement 14.

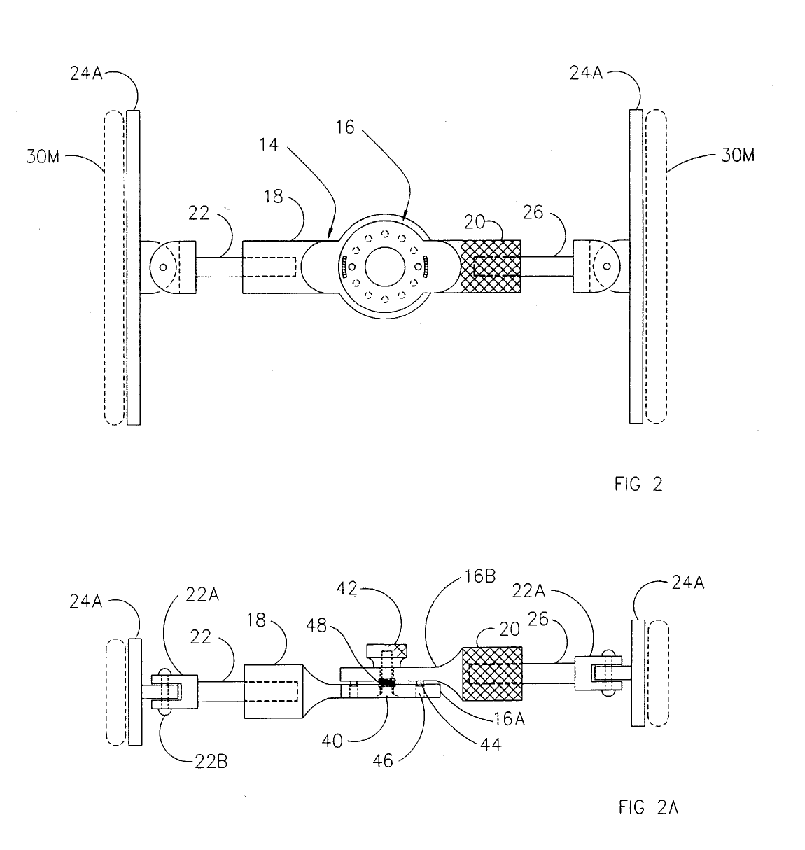

As FIGS. 2 and 2A in conjunction shows, the tensioner arrangement 14 comprises mainly of a goniometer 16 attached to a pair of cylindrical members 18 and 20. The goniometer 16 consists of a pair of discs 16A and 16B which are engageable to each other by such means as a fastener screw 40 in conjunction with a securing knob 42.

Furthermore, the goniometer's disc 16B includes a plurality of non removable engaging pins 44, which are matchable and correspond with a plurality of cylindrical holes 46 provided in the goniometer's disc 16A.

As best seen in FIG. 2A a coil spring 48 is provided between the goniometer's discs 16A and 16B which are constructed in such a manner as to facilitate angle readings between the coupling members 10 and 12. Alternatively, the goniometer 16 may not be used at all if not needed. As seen in FIG. 2 in conjunction with FIG. 2A the cylindrical member 18 is constructed in such a manner as to have one of its longitudinal end attached to goniometer's disc 16A while the opposite longitudinal end receives an accommodating male clockwise threaded member 22. The threaded member 22 is pivotably connected by an end bracket 22A to a medial elongated restraining bar or stiffener 24A.

Furthermore, the cylindrical member 20 is constructed in such a manner as to have one of its longitudinal end attached to the goniometer disc 16B while the opposite longitudinal end receives an accommodating male counter clock wise threaded member 26. The male threaded member 26 is pivotably connected with the end bracket 22A to the medial stiffener 24A. The tensioner arrangement 14, the goniometer 16, the stiffener 26 and 26A may be constructed of any suitable material such as metal, plastic or combination of these materials.

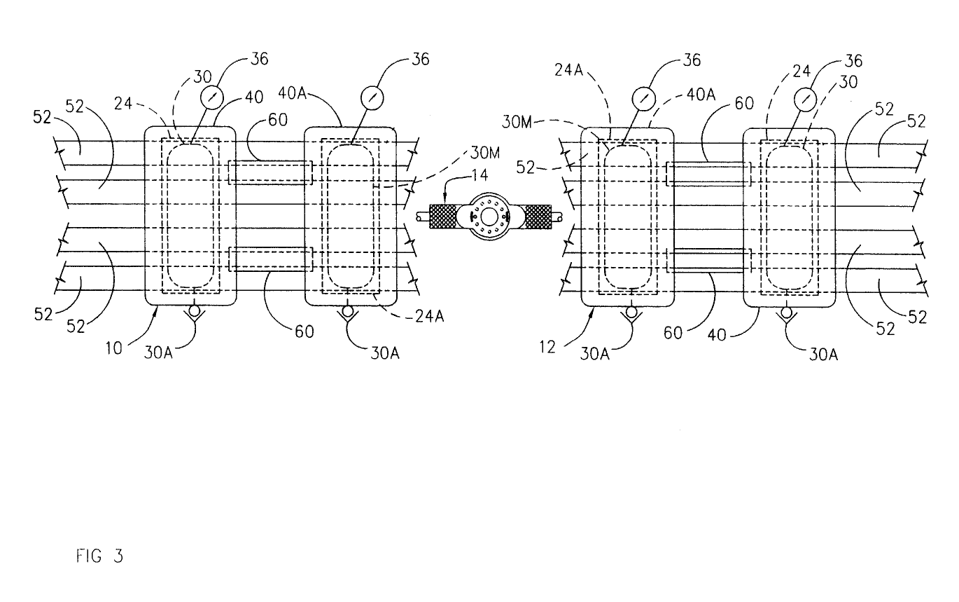

As will be apparent on examining FIG. 1 in conjunction with FIG. 3 an inflatable cushion or element 30M is interposed between the medial stiffeners 24A and the corresponding surface of the lower extremity. The inflatable element 30M is contained in a pocket included in the coupling member structures 10 and 12. Furthermore, a similar inflatable element 30 is interposed between the lateral stiffener 24 and the corresponding surface of the lower extremity.

The stiffeners 24 and 24A are shaped in such a manner as to accommodate the functional size and shape of the element 30. The inflatable elements 30 and 30M include each an air flow directional check valve 30A which allows to be pressurized by a conventional inflation device.

Furthermore, each of the inflatable elements 30 and 30M may include air pressure control means such as air pressure gauges 36, with the purpose to avoid over inflation and to provide accurate readings of the muscular energy contraction.

As shown in FIG. 1 and FIG. 3 in conjunction, each of the coupling members or braces 10 and 12 are constructed in such a manner as to include a pair of half sleeves 40 and 40A. The half sleeve 40 is configured to be applied on the lower extremity in apposition to the half sleeve 40A. In addition as FIG. 3 indicates the half sleeves 40 and 40A are attached posteriorly to each other by such means as a plurality of elastic straps 60.

The elastic straps 60 allow a proper positioning of the lateral half sleeve 40 in apposition to the medial half sleeve 40A when the device is applied on the lower extremity.

An arrangement of hook and loop fasteners 52 is included in order to assist the coupling members 10 and 12 to be secured around the lower extremities of a person.

As FIG. 1 shows the present device is removably applied on the lower extremities of a person in a circumscribed manner.

The hook and loop fasteners 52 may be employed in securing the coupling members 10 and 12 comfortably around the lower extremities. By using a conventional inflation device the inflatable elements 30 and 30M may be inflated through the directional check valve 30A to a predetermined air pressure. The present device may be employed in the rehabilitation process of the lower extremities in an essentially isometric manner.

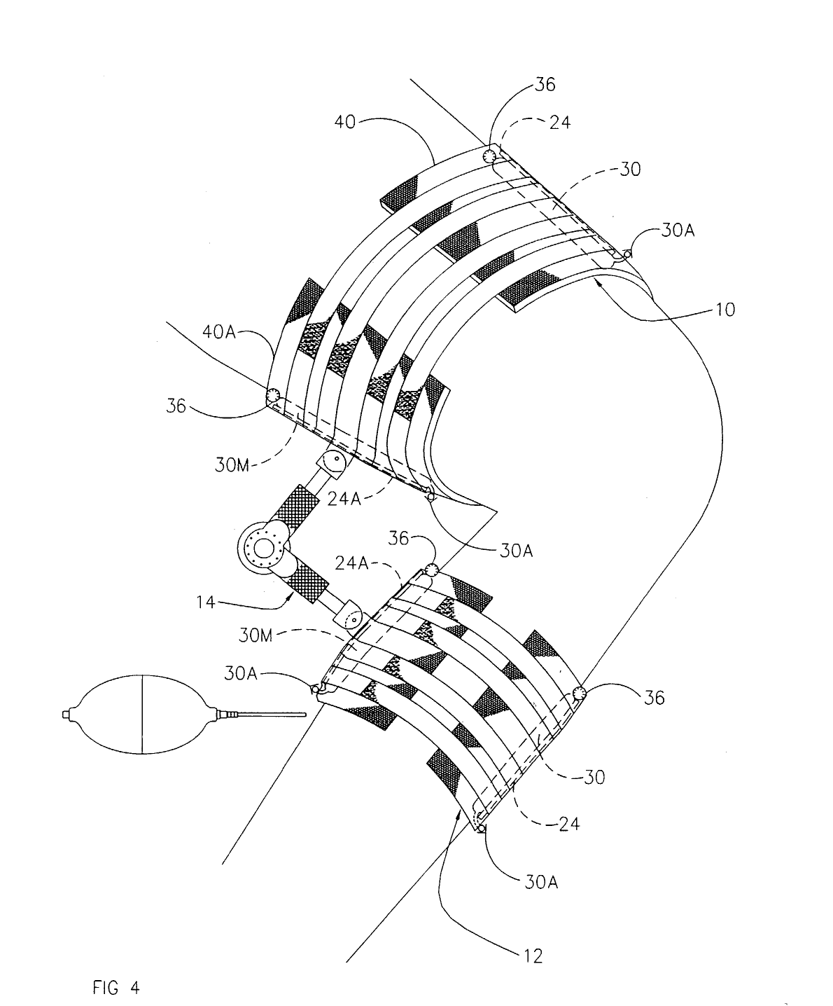

Alternatively FIG. 1 in conjunction with FIG. 4 indicates that the present device may be employed as a brace arrangement, to assist in decreasing contractures of the lower extremities in the rehabilitation process.

The goniometer arrangement 16 may be set to a desired angle. Also by rotating the cylindrical members 18 and 20 either clockwise or counterclockwise, will enable a linear movement of the associated male screw member 22 attached to the brace 10 and the corresponding male screw member 26 attached to the brace 12. Consequently a desired therapeutic position of the lower extremities may be facilitated.

Furthermore as the brace configuration shows in FIG. 4, and similarly to the operation process described above, the present device may be employed for exercising the flexors and extensors muscle of an affected lower extremity in an isometric manner. In addition, it is understood that the present invention may be adopted to fit other body parts such as the upper extremities of a person. Although the description above contains many specificities, these should not be construed as limiting the scope of the invention but merely as providing illustrators of the preferred embodiment of this invention. Thus, the scope of the invention should be determined by the appended claims and their legal equivalents rather than by the examples given.

I claim:

1. A therapeutic lower extremity device comprising:
a. A pair of coupling members including means for securing said coupling members on at least a lower extremity of a person.
b. A tensioner structure having means that enable said coupling members to be positioned in a facilitating therapeutic position.
c. A plurality of air inflatable elements including a plurality of air flow directional check valves.

2. Therapeutic lower extremity device according to claim 1 wherein the coupling members are interconnected by a tensioner structure, said structure including an arrangement of means for an adjustable linear displacement between said coupling members.

3. The therapeutic lower extremity device according to claim 1 wherein each of the air inflatable elements includes a connected air pressure gauge.

4. The therapeutic lower extremity device according to claim 1 further including a goniometer arrangement connected to the tensioner structure, said goniometer arrangement enables angle readings between the coupling members when applied on the patient.

5. A therapeutic lower extremity exercise device comprising:
a. A pair of coupling members
b. A tensioner structure associated with said coupling members
c. A pair of air inflatable elements in each said coupling member.

6. The therapeutic lower extremity exercise device of claim 5 wherein each said coupling member includes a medial and a lateral half shell structure interconnected with a plurality of connecting straps

7. The therapeutic lower extremity exercise device of claim 5 wherein the tensioner structure is pivotably connected to the medial side of the each coupling member.

8. The therapeutic exercise device according to claim 1 wherein the coupling members are constructed in such a manner as to be applied on a upper extremity of a person.
